(19) United States
(12) Patent Application Publication
(10) Pub. No.: US 2021/0299427 A1
(43) Pub. Date: Sep. 30, 2021

(54) FLUID COUPLING

(71) Applicant: Gentherm Medical LLC, Cincinnati, OH (US)

(72) Inventor: Andrew Bradley, Middletown, OH (US)

(21) Appl. No.: 17/204,243

(22) Filed: Mar. 17, 2021

Related U.S. Application Data
(60) Provisional application No. 63/001,753, filed on Mar. 30, 2020.

(51) Int. Cl. A61M 39/10

(52) U.S. Cl. CPC A61M 39/1011; A61F 7/0097; A61M 2039/1033

(57) ABSTRACT

A coupling that includes a male part and a female part. The male part has a distal most end, and a retaining projection having a retaining surface located proximal to the distal-most end of the male part. The female part has a retaining recess having a retaining top edge. The male part is configured to be received into the female part, and the retaining top edge of the retaining recess is configured to engage the retaining surface of the retaining projection. The male part may be connected to a medical hose and the female part may be connected to a medical device, or vice versa, wherein the medical device is a source of fluid, a thermal blanket, a pad, or a mattress.

### PRIORITY

This application claims priority to U.S. 63/001,753 filed on Mar. 30, 2020, the entire disclosure of which is hereby incorporated by reference herein for all purposes.

### FIELD

These teachings relate to a coupling for connecting together two components, such as a device and a hose.

### BACKGROUND

A coupling may be used in a variety of applications to connect together two components. For example, a coupling may be used to connect together a hose and a device, which may be a fluid supply or fluid receiver.

A coupling may generally include a first part that is fixed or attached to the hose and a second part that is fixed or attached to the device. These two parts can be joined together by way of, for example, threading the two parts together and/or inserting one part into the other part. While a variety of couplings available today may be suitable for their intended purpose, improvement in the art may be desired.

For example, some couplings require engaging a plurality of threads between the two parts to establish the connection. This may increase the time required to connect and/or disconnect the coupling, which in certain applications may be unfavorable, such as in emergency medical situations. Moreover, should a user or operator fail to engage at least a minimum number of threads between the two parts, a risk exists of a fluid leak between the two parts. Some couplings are configured with a reverse thread, which may make joining together the two parts awkward or tedious, especially if an operator is not aware the coupling is a reverse-thread design. Accordingly, it may be desirable to have an improved coupling that can overcome at least some of these deficiencies.

### SUMMARY

These teachings provide a coupling that includes two parts, namely a first part and a second part. The first part may refer to a female part and the second part may refer to a male part, or vice versa. The male part has a distal most end, and a retaining projection having a retaining surface located proximal to the distal-most end thereof. The female part has a retaining recess that includes a retaining top edge. The male part is configured to be received into the female part, and the retaining top edge of the retaining recess is configured to engage the retaining surface of the retaining projection.

### BRIEF DESCRIPTION OF THE DRAWINGS

FIG. 1 illustrates an exemplary device and an exemplary hose connected together with a coupling according to these teachings.

FIG. 2 illustrates an exemplary device and an exemplary hose connected together with a coupling according to these teachings.

FIG. 3A is a perspective view of a first or female part of a coupling.

FIG. 3B is another perspective view of the first or female part of the coupling.

FIG. 3C is a sectional view of the first or female part of the coupling, taken along lines 3C-3C in FIG. 3A.

FIG. 3D is detailed view of the portion identified 3D of the female part in FIG. 3C.

FIG. 4A is a perspective view of a second or male part of the coupling.

FIG. 4B is another perspective view of the second or male part of the coupling.

FIG. 4C is another perspective view of the second or male part of the coupling.

FIG. 4D is a front view of the second or male part of the coupling.

FIG. 5A is a perspective view of a step of connecting together the first part and the second part of the coupling.

FIG. 5B is a sectional view of a step of connecting together the first part and the second part of the coupling.

FIG. 5C is a perspective view of the first part and the second part of the coupling connected together.

FIG. 5D is a sectional view of the first part and the second part of the coupling connected together.

FIG. 6 is a perspective view of a coupling according to these teachings.

FIG. 7A is a perspective view of a first or female part of the coupling of FIG. 6.

FIG. 7B is a sectional view of the first or female part of FIG. 7A.

FIG. 8A is a perspective view of the second or male part of the coupling of FIG. 6.

FIG. 8B is a side view of the second or male part of the coupling of FIG. 6.

FIG. 8C is a sectional view of the second or male part of FIG. 8B.

FIG. 9 is a perspective view of a coupling according to these teachings.

FIG. 10A is a perspective view of a first or female part of the coupling of FIG. 9.

FIG. 10B is a sectional view of a portion of the first or female part of FIG. 10A

FIG. 11 is a perspective view of the second or male part of the coupling of FIG. 9.

FIG. 12 is a detailed view of retaining projection of the second or male part of the coupling of FIG. 11.

FIG. 13A is a perspective view of a step of connecting together the first part and the second part of the coupling of FIG. 9.

FIG. 13B is a perspective view of a step of connecting together the first part and the second part of the coupling of FIG. 9.

FIG. 13C is a detailed view of a portion of the first part and the second part of the coupling of FIG. 9 connected together.

FIG. 14 is a perspective view of a coupling according to these teachings.

FIG. 15A is a perspective view of a first or female part of the coupling of FIG. 14.

FIG. 15B is a front view the first or female part of the coupling of FIG. 14.

FIG. 15C is a sectional view of a portion of the first or female part of FIG. 15A.

FIG. 16A is a perspective view of the second or male part of the coupling of FIG. 14.

FIG. 16B is a front view of the second or male part of the coupling of FIG. 14.

FIG. 16C is a sectional view of a portion of the first or female part of FIG. 16A.

FIG. 17A is a perspective view of a step of connecting together the first part and the second part of the coupling of FIG. 14.

FIG. 17B is a perspective view of a step of connecting together the first part and the second part of the coupling of FIG. 14.

FIG. 17C is a perspective view of the first part and the second part of the coupling of FIG. 14 connected together.

### DETAILED DESCRIPTION

The device described herein may be virtually any device. The device may be used in medical applications—i.e., the device may be a medical device. The device may be used in automotive, aerospace, and the like. For example, the device may be a source of a fluid, which may include, for example: a source of air, a source of liquid, or both. The air and/or the fluid may be conditioned. This means that a temperature of the air and/or liquid may be warmer than an ambient temperature, cooler than an ambient temperature, or generally the same as ambient.

The device may be a source of fluid. For example, the source may be or may include a fluid reservoir, container, a bag, or other fluid storage medium. The source may include a pump or other device for moving fluid from the device to and through the hose. The fluid may be moved through the hose via gravity.

The device may be a destination. A destination may be a device to which fluid is supplied. For example, the destination may be supplied with a fluid via one or more hoses. The destination may be a thermal blanket, pad, pillow, or mattress. The destination may be a thermal seat, a seat insert, or a component thereof. The destination may be a fluid reservoir, container, or other fluid storage medium. The fluid may be transferred to and/or from the device and/or source via one or more hoses.

The hose may be a duct, pipe, hose, pipeline, passageway, conduit, or other device for connecting to any device. The hose may function to permit the transfer of a fluid between two or more devices. The male part or the female part of the coupling may be connected or attached to the hose. For example, a proximal part of the male part or the female part may be removably attached to the hose via one or more fasteners or adhesives. For example, the female part or the male part may be threaded onto the hose so that the part can be removed from the device when not in use or to be attached to other devices. For example, a proximal part of the male part or the female part may be permanently attached to the hose via one or more fasteners (i.e., clamps, crimps, welds, pins, snaps, threads, magnets) or adhesives). In some instances, the male part or the female part may be integrally formed with the hose or with a portion of the hose.

The male part or the female part of the coupling may be connected or attached to the device. For example, a proximal part of the male part or the female part may be removably attached to the device via one or more fasteners (i.e., clamps, crimps, welds, pins, snaps, threads, magnets) or adhesives. For example, the female part or the male part may be threaded onto the device so that the part can be removed from the device when not in use or to be attached to other devices. For example, a proximal part of the male part or the female part may be permanently attached to the device via one or more fasteners or adhesives. In some instances, the male part or the female part may be integrally formed with the device.

The male part or the female part may be attached to the hose so that the hose can move (longitudinally, laterally, proximally, distally, axially, and/or rotationally) relative to the male part or the female part. This may advantageously assist with assembling and/or disassembling the part to the hose and/or attaching the two parts of the coupling together.

The coupling comprises one or more male parts. The male part may be the part of the coupling that is at least partially received into the female part. The male part may be made of a suitable material, such as a plastic, such as polypropylene, nylon, vinyl, acetal, polycarbonate, polyethylene, Polyethylene terephthalate glycol (PETG), polyethylene terephthalate (PET), Polystyrene, high impact Polystyrene (HIPS), etc. However, some male parts may also or may instead comprise metal, like stainless steel, titanium, tantalum, aluminum, etc. The male part may be injection molded form one or more materials, cast, 3D printed, or a combination thereof. The male part may be configured to be a single-use or disposable component. The male part may be configured to be reusable for a plurality of procedures. The male part may be autoclavable to ensure sterility between procedures. The male part may be configured for attaching to the device or the hose, or the male part may be configured to attach to both male and female parts, depending on the application.

The male part and/or the hub may include the one or more retaining projections or retainers. The retaining projections or retainers may be equally or unequally spaced about or around a the hub, for example, around a circumference of the hub. Unequally spaced apart means that a gap between adjacent retaining projections or retainers is not uniform around the male part. The retaining projections or retainers may have a length. The length of the retaining projections or retainers may be slightly smaller than a length of the retaining recesses defined in the female part into which the retaining projections or retainers are received. The length of the retaining projection and/or recess may be defined along a line that is generally parallel to the center longitudinal axis of the male part or female part. The retaining recesses may have a distal-most end that is generally aligned with the distal most end of the male part. On the other hand, the distal-most end of the retaining recess may be proximal to the distal most end of the male part or hub. The proximal end of the retaining recesses may be the top retaining surface identified at 156. The retaining projection, the proximal or distal end of the retaining projection or a combination thereof may include one or more clips, tabs, undercuts, or other engaging features for engaging the corresponding retaining recesses or the female part to create/maintain a connection therebetween.

The one or more retaining projections or retainers may be a clip or other feature or element that is added or assembled to the male part after the male part is formed or made. The one or more retaining projections or retainers may be disposed along an outside surface or portion of the male part, collar, hub, or a combination thereof. The one or more retaining projections or retainers may be disposed along an inside surface or portion of the male part, collar, hub, or a combination thereof.

The one or more retaining projections or retainers may be a clip, prong, finger, undercut, protuberance, projection, or the like. The one or more retaining projections or retainers may be flexible, biasable, deflectable, and/or movable so that the one or more retaining projections or retainers can engage a corresponding one or more retaining, engaging, or locking feature on the female part.

The one or more retaining projections or retainers may be retained by one or more features of the female part. The one or more features of the female part may engage, contact, abut, or be located near or adjacent to one or more top, side, or bottom surfaces of the one or more retaining projections or retainers to axially and/or rotationally lock the male and female parts together.

In some configurations, one or more features, retaining projections, or retainers described with respect to the male part may be added to the female part and/or one or more feature, elements, or retaining recesses or elements on the female part may be added to the male part.

The male part may be rotated relative to the female part for the one or more retaining projections to engage the one or more retaining recesses. The female part may be rotated relative to the male part for the one or more retaining projections to engage the one or more retaining recesses. Both the male and female parts may be rotated (in opposite directions relative to each other) for the one or more retaining projections to engage the one or more retaining recesses. The rotation may be on the order of about 5 degrees or more, 15 degrees or more, 25 degrees or more, 45 degrees or more, 60 degrees or more, 90 degrees or more, 120 degrees or more, 150 degrees or more, 180 degrees or more, 270 degrees or more, 360 degrees or more. The rotation may be on the order of about 360 degrees or less, 270 degrees or less, 180 degrees or less, 150 degrees or less, 120 degrees or less, 90 degrees or less, 60 degrees or less, 45 degrees or less, 25 degrees or less, 15 degrees or less, 5 degrees or less.

The male part may include one or more collars. The collar may have an outer diameter that is greater than an outer diameter of the hub and/or the one or more retaining projections. The collar may be used by an operator to grab, handle, and/or rotate the male piece during assembly/disassembly of the coupler.

The male part, the collar, and/or the hub may include one or more gripping ribs or projections. The one or more gripping ribs or projections may aid in gripping, rotating and/or handling of the male part and/or the coupler. The one or more gripping ribs or projections may be raised areas, bumps, ridges, hills, nubs, etc., extending from the collar and/or any outer surface of the male part. One or more valleys, recesses, voids, etc. may be provided between the ribs or projections. The one or more gripping ribs or projections may be aligned with the one or more retaining projections along an axis. The axis may be generally parallel to the longitudinal axis of the coupling and/or the male part. The one or more gripping ribs may be provided between two adjacent gripping projections.

The male part may include a proximal portion. The proximal portion may be attached to the device or hose. The proximal portion may include one or more ribs. The one or more ribs may function as a locator so that the male part is rotationally attached to the device or hose in one or more configurations. The one or more ribs may be threads for threadably engaging the respective hose or device. An outer diameter of the proximal portion may be generally the same size as an outer diameter of the collar, the hub or distal portion or both. An outer diameter of the proximal portion may be generally smaller than an outer diameter of the collar, the hub or distal portion or both. An outer diameter of the proximal portion may be generally larger than an outer diameter of the collar, the hub or distal portion or both.

The male part may include one or more seals or gaskets. The one or more seals or gaskets may function to create a fluid seal with the corresponding mating components of the female part to form a fluid seal therebetween to restrict or prevent fluid from leaking therebetween.

The male part may include one or more ribs, threads, snaps, retainers, or other features for connecting to the device, hose, or both. The one or more ribs, threads, snaps, retainers, or other features may be located on an inner diameter or outer diameter of the hub, collar, proximal portion, distal portion flange, or a combination thereof.

The coupling comprises a female part. The female part may be the part of the coupling into which at least a portion of the male part is received. The female part may be made of a suitable material, such as a plastic. The female part may be made of a suitable material, such as a plastic, such as polypropylene, nylon, vinyl, acetal, polycarbonate, polyethylene, Polyethylene terephthalate glycol (PETG), polyethylene terephthalate (PET), Polystyrene, high impact Polystyrene (HIPS), etc. However, some female parts may also or may instead comprise metal, like stainless steel, titanium, tantalum, aluminum, etc. The female part may be injection molded form one or more materials, cast, 3D printed, or a combination thereof. The female part may be configured to be a single-use or disposable component. The female part may be configured to be reusable for a plurality of procedures. The female part may be autoclavable to ensure sterility between procedures. The female part may be configured for attaching to the device or the hose, or the female part may be configured to attach to both male and female parts, depending on the application. The female part may be made of the same material as the male part or different materials.

The female part and/or the socket comprises one or more receiving recesses. The one or more receiving recesses may be sized to accept at least a portion of the male part, the one or more retaining recesses, retainers, or a combination thereof. This means an opening of the one or more receiving recesses and/or the hub is at least slightly larger than an outer diameter of the portion of the male part and/or the one or more retaining projections or retainers.

The female part includes one or more retaining recesses. One or more retaining recesses may be defined between two adjacent receiving recesses. However, other arrangements are envisioned. For example, the female part may have two or more retaining recesses defined between two adjacent receiving recesses. Of course, the retaining projections on the male part would need to be arranged in a similar format to cooperate with these features in the female part to form the coupling.

The retaining recesses may be equally or unequally spaced about or around the socket. The retaining recesses may have a length. The length of the retaining recesses may be slightly larger than a length of the retaining projections defined in the male part. The length of the retaining recesses may be defined along a line that is generally parallel to the center longitudinal axis of the female part. The retaining recesses may have a distal-most end that is proximal to the distal most end of the hub or female part. The distal end of the retaining recesses may be the top feature identified at 118. The retaining recesses, the proximal or distal end of the retaining recesses or a combination thereof may include one or more clips, tabs, undercuts, or other engaging features for engaging the corresponding retaining projections or the male part to create/maintain a connection therebetween.

The female part may include an outer surface. The outer surface may be used by an operator to grab, handle, and/or rotate the male piece during assembly/disassembly of the coupler. The outer surface of the female part may include one or more gripping ribs or projections. The one or more gripping ribs or projections of the female part may aid in gripping, rotating and/or handling of the female part and/or the coupler. The one or more gripping ribs or projections of the female part may be raised areas, bumps, ridges, hills, nubs, etc. One or more valleys, recesses, voids, etc. may be provided between the ribs or projections. The one or more gripping ribs or projections of the female part may be formed by the structure corresponding to the one or more receiving and/or retaining recesses. The one or more gripping ribs or projections of the female part may be a scalloped surface.

The female part may include a proximal portion. The proximal portion may be attached to the device or hose. The proximal portion may include one or more ribs. The one or more ribs may function as a locator so that the female part is rotationally attached to the device or hose in one or more configurations. The one or more ribs may be threads for threadably engaging the respective hose or device. An outer diameter of the proximal portion may be generally the same size as an outer diameter of the socket. An outer diameter of the proximal portion may be generally smaller than an outer diameter of the socket. An outer diameter of the proximal portion may be generally larger than an outer diameter of the socket.

The female part may include one or more seals or gaskets. The one or more seals or gaskets may function to create a fluid seal with the corresponding mating components of the male part to form a fluid seal therebetween to restrict or prevent fluid from leaking therebetween.

The female part may include one or more ribs, threads, snaps, retainers, or other features for connecting to the device, hose, or both. The one or more ribs, threads, snaps, retainers, or other features may be located on an inner diameter or outer diameter of the any portion of the female part.

Any of the elements or features disclosed herein may be duplicated, eliminated, copied, or rearranged onto other areas of the male and/or female part, without departing from the scope of the disclosure. Moreover, while certain elements are described as being part of the male part, it is understood that some of those elements may be also located on the female part or may instead be located on the female part. Similarly, while certain elements are described as being part of the female part, it is understood that some of those elements may be also located on the male part or may instead be located on the male part. Elements disclosed as being part of one embodiment of the coupling, male part, and/or female part may be combined, shared, or substituted with elements that are part of another embodiment of the coupling, male part, and/or female part.

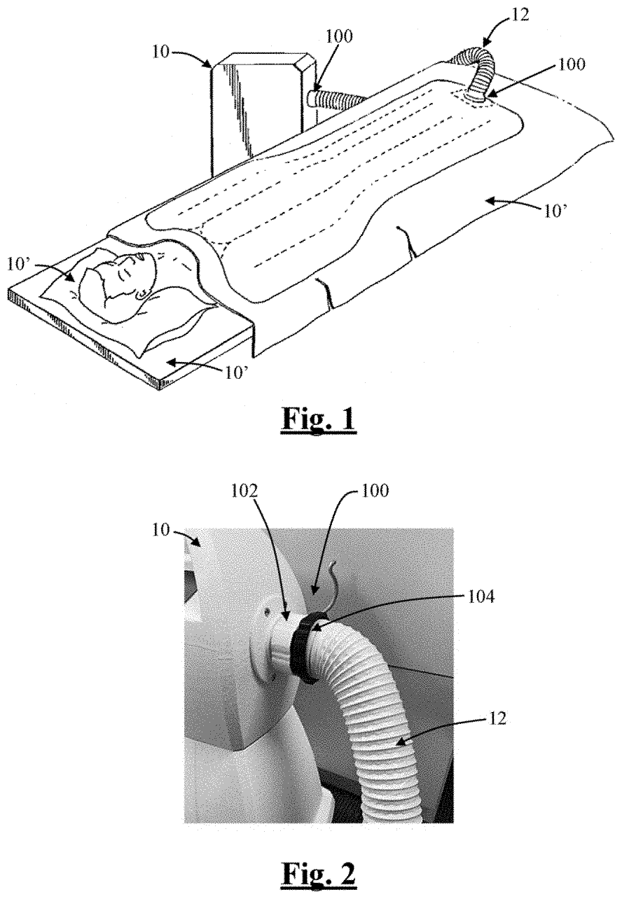

FIG. 1 illustrates two exemplary parts connected together with a coupling 100 according to these teachings. One of the parts is an exemplary device 10 and the other part is an exemplary hose 12. The device 10 may be a fluid source. The hose 12 may be configured to transport or provide a passageway for one or more fluids to move between the device 10 and another device 10′, which may be a fluid destination such as a thermal blanket. The device 10′ may be connected to the other end of the hose 12 with a coupling 100 according to these teachings. The device 10′ may also be a thermal pad or mattress.

FIG. 2 illustrates a hose 12 connected to a device 10 via a coupling 100 according to these teachings. The coupling 100 comprises a first or female part 102 and a second or male part 104. The first or female part 102 is connected to the device 10 and the second or male part 104 is connected to the hose 14. In other configurations within the scope of this disclosure, the first or female part 102 may be connected to the hose 12 and the second or male part 104 may be connected to the device 10.

Various couplings and parts of couplings are disclosed in this application. It is understood that any of the couplings described and/or illustrated herein may be used in place of the coupling 100. Moreover, one or more features, elements, and/or parts of the coupling illustrated and/or described herein may be combined, omitted, substituted, duplicated, eliminated, and/or modified to form a suitable connector for connecting together the hose 12 and device 10.

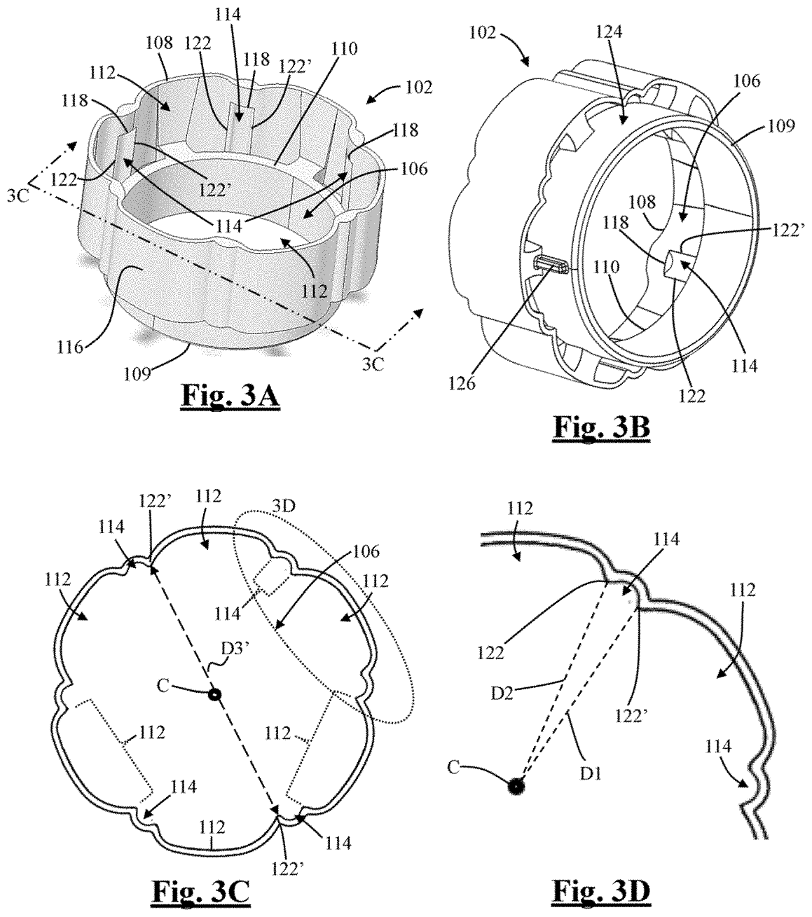

FIGS. 3A-3D illustrate the first or female part 102 of the coupling 100. The female part 102 comprises a socket 106. The socket 106 has a generally circular wall 116 that extends around a center C of the female part 102. The socket 106 is defined between a leading edge 108 of the female part 102 and a contact wall or ledge 110. The leading edge 108 may be the distal-most end of the female part 102. The contact wall or ledge 110 extends inwardly from the wall 116 towards the center C of the female part 102 and is located in a region between the leading edge or distal-most end 108 of the female part 102 and a trailing edge 109 (i.e., a proximal-most end) of the female part 102.

The female part 102 may comprise one or more receiving recesses 112. The receiving recess 112 may be disposed or defined in the wall 116 of the socket 106. The receiving recesses 112 may be equally spaced around the circumference or perimeter of the socket 106. Alternatively, the receiving recesses 112 may be unequally spaced around the circumference or perimeter of the wall 116 of the socket 106.

The female part 102 may comprise one or more retaining recesses 114. The retaining recesses 114 may be disposed or defined in the wall 116 of the socket 106. The retaining recesses 114 may be disposed or defined between the contact wall or ledge 110 and a retaining top edge 118 of the retaining recess 114 that is defined between the contact wall or ledge 110 and the leading edge or distal-most end 108 of the female part 102.

One or more of the retaining recesses 114 may be canted or turned relative to the center C of the female part 102. In other words, a retaining recess 114 may be defined between opposing retaining side edges 122, 122′. One of the side edges 122′ extends or is located closer towards the center C of the female part 102 than the other side edge 122. Stated another way, referring specifically to FIG. 3D, the distance D1 between side edge 122′ and the center C is less than the distance D2 between the side edge 122 and the center C. Accordingly, for a respective retaining recess 114, the edge 122′ that is closer towards the center C may be described as being further clockwise (which may be referred to as a “clockwise edge”) relative to the other edge 122 which may be referred to as a “counterclockwise edge”). And, accordingly, the edge 122 (i.e., the “counterclockwise edge”) that is farther away from the center C may be described as being further counterclockwise than the other edge 122′ (i.e., the “clockwise edge”).

A retaining recess 114 may be defined between two adjacent receiving recesses 112. The retaining recesses 114 may be equally spaced around the circumference or perimeter of the wall 116 of the socket 106. Alternatively, the retaining recesses 114 may be unequally spaced around the circumference or perimeter of the socket 106.

An outer surface of the female part 102 or the wall 116 of the socket 106 may be scalloped or clover shaped. In other words, the outer radial edge or surface or wall of the socket or female part 102 may include repeating pattern of humps or curves and notches therebetween defined by the receiving recesses 112 and the retaining recesses 114, respectively.

The female part 102 comprises a trailing part 124. The trailing part 124 may be the section or portion of the female part 102 that connects with or engages one of the devices 10, 10′ or the hose 12. The trailing part 124 may be the proximal part or surface or edge of the female part 102. The female part 102 may include a locator or rib 126 disposed at or on the trailing part 124 to assist with locating the female part 102 on the device 10, 10′ or hose 12, to assist with retaining the female part 102 on the device 10, 10′ or hose 12, or both.

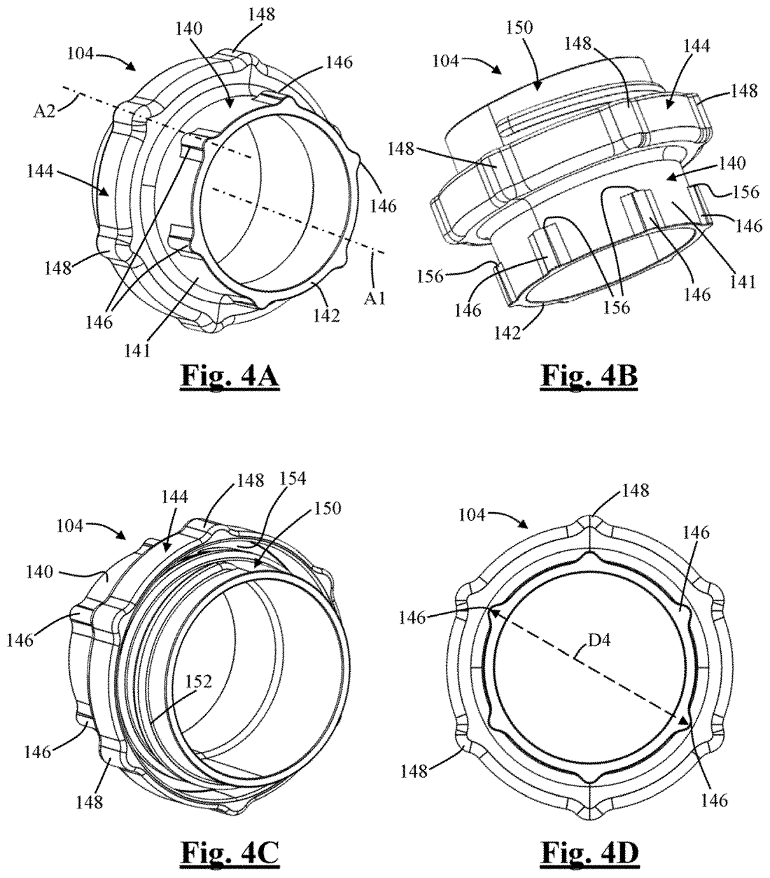

FIGS. 4A-4D illustrate the second or male part 104 of the coupling 100. The male part 104 comprises a hub 140. The hub 140 is defined between a leading edge 142 of the male part 104 and a collar 144. The leading edge 142 may be the distal-most end of the male part 104.

The male part 104 may comprise one or more retaining projections 146. The one or more retaining projections may be disposed or extend from an outer surface 141 from the hub 140. The retaining projections 146 have having a length (or extend between) a top retaining surface 156 and the leading edge 142 of the male part 104. The top retaining surface 156 extends away from an outer surface 141 of the hub 140 and is defined between the leading edge 142 of the male part 104 and a collar 144.

The retaining projections 146 may be disposed around a circumference or perimeter of the hub 140. The retaining projections 146 may be equally spaced around the circumference or perimeter of the hub 140. Alternatively, the retaining projections 146 may be unequally spaced around the circumference or perimeter of the hub 140. Preferably, the retaining projections 146 are arranged or spaced on the hub 140 in a manner that generally or substantially corresponds to the arrangement or spacing of the receiving recesses 112 and retaining recesses 114 on the female part 102 (FIGS. 3A-3D).

The male part 104 may comprise one or more gripping projections 148 disposed or extending from the collar 144. The gripping projections 148 may be disposed around a circumference or perimeter of the collar 144. The gripping projections 148 may be equally spaced around the circumference or perimeter of the collar 144. Alternatively, the gripping projections 148 may be unequally spaced around the circumference or perimeter of the collar 144. One or more of the gripping projections 148 may be aligned with a corresponding retaining projection 146 along an axis A2. The axis A2 may be generally parallel to an axis A1 along which the male part 104 extends.

The male part 104 comprises a trailing section 150. The trailing section 150 may be the section of the male part 104 that connects with or engages one of the devices 10, 10′ or hose 12. The trailing section 150 may be a proximal end or portion of the male part 104. The male part 104 may include a locator or rib 152 to assist with locating male part 104 on the device 10, 10′ or hose 12, to assist with retaining male part 104 on the device 10, 10′ or hose 12, or both.

The male part 104 may include an undercut region 154. The undercut region 154 may be configured to accept a portion or section of the corresponding device 10, 10′ or hose 12 to which the male part 104 is attached or connected to. The undercut region 154 may extend at least partially under the collar 144. For example, a portion of the hose 12 may be threaded onto the locator or rib 152 and extend into the undercut region 154.

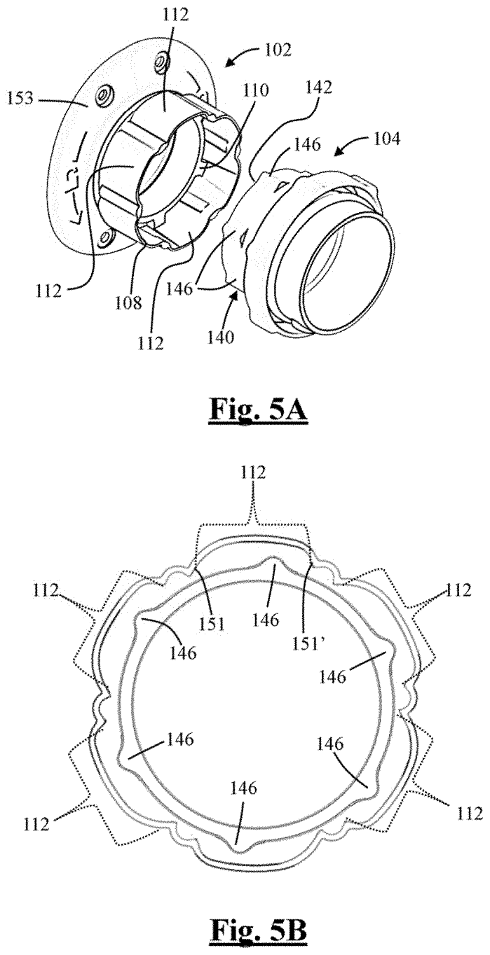

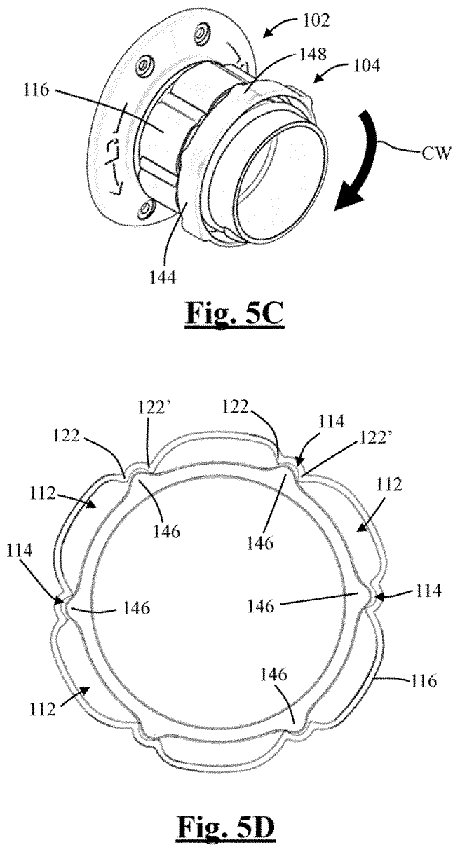

FIGS. 5A-5D illustrate a method of connecting together the female part 102 and the male part 104. It is understood that the following method steps can be performed in virtually any order. Moreover, one or more of the following method steps can be combined with other steps; can be omitted or eliminated; can be repeated; and/or can separated into individual or additional steps.

The proximal potion or section of female part 102 may be connected to a device 10 or 10′ (1 and 2) and the proximal portion or section of the male part 104 may be connected to hose 12 (FIGS. 1 and 2), or vice versa.

Referring to FIGS. 5A and 5B, the leading or distal-most edges 108, 142 of the female and male parts 102, 104 can be bought together so that the male part 104 or the hub 140 of the male part 104 is received into the female part 102 or the socket 106 of the female part 102. More specifically, the female and male parts 102, 104 are bought together so that the retaining projections 146 on the hub 140 are received into or within the corresponding receiving recesses 112 within the socket 106. The parts 102, 104 may be brought together or towards each other until the leading or distal-most edge 142 of the male part 104 bottoms out, abuts, or contacts the contact wall or ledge 110 of the female part 102 (See also FIG. 3A). The female part 102 may include a flange 153 to aid or assist with connecting the female part 102 to the device 10, 10′ or hose 12. In other configurations, the male part 104 may also or may instead have the flange 153.

In the partially coupled position illustrated in FIG. 5B, the male and female parts 102, 104 remain axially moveable or separable relative to each other. Also, in this illustrated partially assembled position of FIG. 5B, the male part 104 may be rotated relative to the female part 102. More specifically the retaining projections 146 of the male part 104 can move or rotate between the corresponding edges 151, 151′ of the receiving recesses 112.

Referring now to FIGS. 5C and 5D, the method includes rotating the male part 104 in a clockwise direction CW (and/or rotating the female part 102 in an opposing counterclockwise direction) until the retaining projections 146 contact and then pass over the retaining side edges 122 (i.e., the counterclockwise located retaining side edge). While the male part 104 is being turned in a clockwise direction CW and while the retaining projections 146 are in contact with and passing over the retaining side edge 122, the wall 116 of the socket 106 may at least partially and deform or flex. After the retaining projections 146 are rotated past the retaining side edge 122 and the retaining projections 146 are located within the retaining recesses 114, the wall 116 may resiliently spring back to its original shape. The retaining projections 146 are then locked or retained between the two retaining edges 122, 122′ of the retaining recesses 114 in FIG. 5D.

In the coupled position illustrated in FIG. 5D, the retaining projections 146 are locked or retained between the two retaining edges 122, 122′ preventing further clockwise CW rotation of the male part 104 relative to the female part 102. This is because the retaining side edge 122′ is located closer to the center C than the other retaining side edge 122 (refer to FIG. 3D and corresponding discussion). Moreover, referring to FIGS. 3C and 4D, the length or size of the opening D3 between opposing side edges 122′ (FIG. 3C) is smaller than the length or size D4 of the male portion 104 between opposing retaining projections 146 (FIG. 4D), which prevents the male portion 104 from further clockwise CW rotation.

Also, in the coupled position illustrated in FIG. 5D axial separation of the two parts 102, 104 is restricted or prevented by way of the top retaining edges 118 (FIGS. 3A/3B) acting on the top retaining surfaces 156 of the retaining projections 146 (FIG. 4B).

The rotation required for the retaining projections 146 to move from the receiving recess 112 to the retaining recess 114 may be about 15 degrees. In other words, for a respective retaining projection 146 to move or rotate past the wall or edge 151′ (FIG. 58) and into the retaining recess 114 may be about 15 degrees.

While moving past the retaining side edge 122 and/or being received into the retaining projections 146, the coupling 100 may make an audible sound, such as a click, alerting the operator that coupling 100 is properly connected or engaged together

To unlock or disconnect the coupling 100, the male portion 104 may be rotated in an opposing counterclockwise direction. Doing so, causes the retaining projections 146 to pass over the retaining side edge 122 and move into the receiving recesses 112. Accordingly, the two parts 102, 104 can then be axially separated.

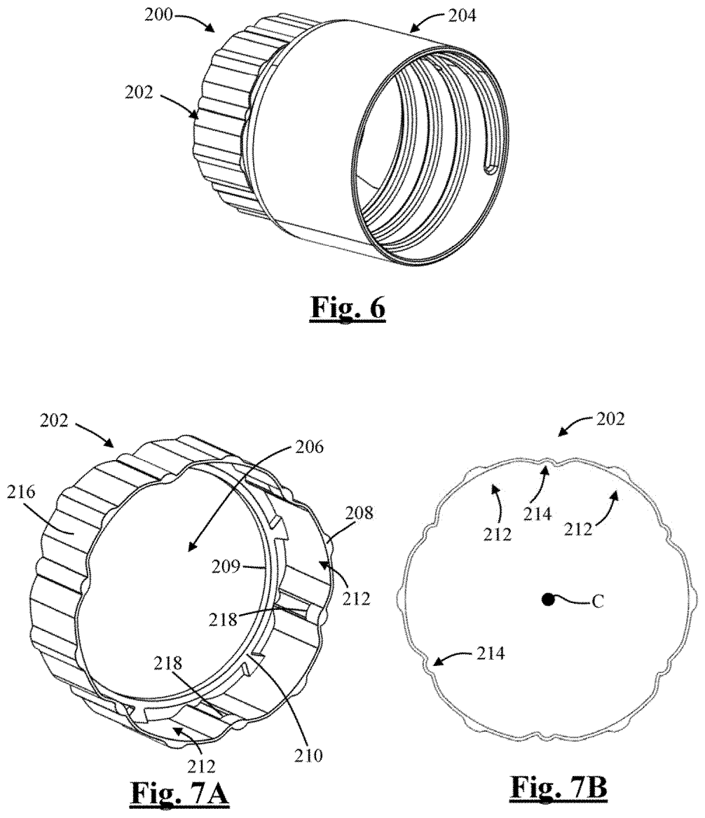

FIG. 6 illustrates a coupling 200 that may be used in place of the coupling 100. It is understood that one or more of the features illustrated and/or described relating to any of the couplings described herein (including those illustrated/described in couplings 100, 300, 400, etc.) may be incorporated into the coupling 200, and vice versa.

Coupling 200 comprises a first or female part 202 and a second or male part 204. With additional reference to FIGS. 1, 2 the first or female part 202 is connected to the device 10 and the second or male part 202 is connected to the hose 14. In other configurations within the scope of this disclosure, the first or female part 202 may be connected to the hose 12 and the second or male part 204 may be connected to the device 10.

FIGS. 7A, 7B illustrate the first or female part 202. The female part 202 comprises a socket 206. The socket 206 has a generally circular wall 216 that extends around a center C of the female part 202. The socket 206 is defined between a leading edge 208 of the female part 102 and a contact wall or ledge 210 defined inside of the socket 206. The leading edge 208 may be the distal-most end of the female part 202. The contact wall or ledge 210 extends inwardly from the wall 216 towards the center C of the female part 202 and is located in a region between the leading edge or distal-most end 208 of the female part 202 and a trailing edge 209 (i.e., a proximal-most end) of the female part 202. The contact wall or ledge 210 thus defines an inner diameter that is smaller than an inner diameter defined by the leading edge 208.

The female part 202 may comprise one or more receiving recesses 212 that may be similar to those described above at 112. The female part 202 may comprise one or more retaining recesses 214 that may be similar to those described above at 214.

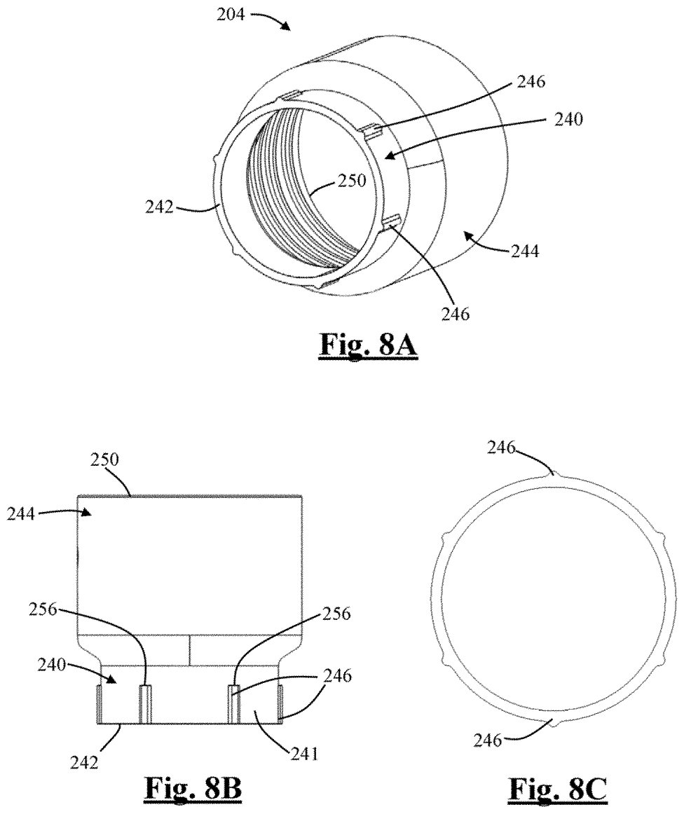

FIGS. 8A-8C illustrate the second or male part 204 of the coupling 200. The male part 204 comprises a hub 240. The hub 240 is defined between a leading edge 242 of the male part 204 and a collar 244.

The male part 204 may comprise one or more retaining projections 246 that may be disposed or extend from an outer surface 241 from the hub 240. The retaining projections 246 have having a length (or extend between) a top retaining surface 256 thereof and the leading edge 242 of the male part 204. The top retaining surface 256 is defined between the leading edge 242 of the male part 204 and a collar 244. The retaining projections 256 may be similar to those described above at 156.

The male part 204 comprises a trailing section 250. The trailing section 250 may be the section of the male part 250 that connects with or engages one of the devices 10, 10′ or hose 12. An inner portion of the trailing section 250 includes a rib 252 or thread onto which the hose 12 or device may be threaded or attached thereto. In some configurations, an outer surface of the trailing section 250 may include the rib 252 or thread. In some configurations, the inner and outer surfaces of the trailing section 250 may include rib 252 or threads which would allow the male part 204 to connect with a variety of hose diameter or sizes.

The two parts together 202, 204 may be connected together in substantially the same manner that the parts 102 and 104 connect together, as described and illustrated above in FIGS. 5A-5D; thus, in the interest of brevity those steps will not be repeated again.

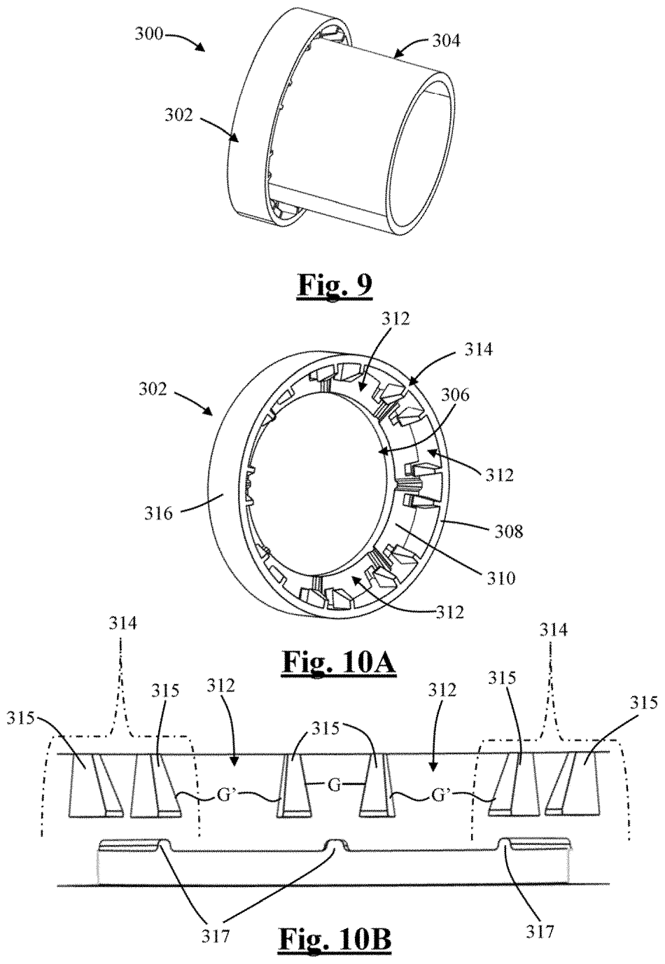

FIG. 9 illustrates a coupling 300 that may be used in place of the coupling 100. The coupling 300 includes a first or female part 302 and a second or male part 304. It is understood that one or more of the features illustrated and/or described relating to any of the couplings described herein (including those illustrated/described in couplings 100, 200, 400, etc.) may be incorporated into the coupling 300, and vice versa.

FIGS. 10A, 10B illustrate the first or female part 302 of the coupling 300. The female part 302 comprises a socket 306. The socket 306 has a generally circular wall 316 that extends around a center of the female part 302. The socket 306 is defined between a leading edge 308 of the female part 302 and a contact wall or ledge 310. The leading edge 308 may be the distal-most end of the female part 302. The contact wall or ledge 310 extends inwardly from the wall 316 towards the center of the female part 302. A diameter defined by an inside of the contact wall or ledge 310 is smaller than a diameter defined by the generally circular wall 316.

The female part 302 may comprise one or more receiving recesses 312. The receiving recess 312 may be disposed or defined in the wall 316 of the socket 306. The receiving recesses 312 may be equally spaced around the circumference or perimeter of the socket 306. Alternatively, the receiving recesses 312 may be unequally spaced around the circumference or perimeter of the socket 306.

The female part 302 may comprise one or more retainers 314. The one or more retainers 314 may be disposed or defined or formed in or extend from the wall 316 of the socket 306. A retainer 314 may comprise one or more upper tabs, projections, or protuberances 315 and one or more lower tabs, projections, or protuberances 317. As illustrated, each of the retainers 314 comprise a pair of upper tabs 315 and a lower tab 317 located between or in a gap G defined between the two upper tabs 315. There may be one or a plurality of the retainers 314 disposed around the socket 306, equally or unequally. Unequally as used throughout this disclosure may mean that the spacing between adjacent retainers 314 is not the same or is random.

A receiving recess 312 is a defined in a region or gap G′ between two adjacent upper tabs 315 that are not part of the same retainer 314. The gap G′ is larger or greater than the gap G between two upper tabs 315 of a common retainer 314.

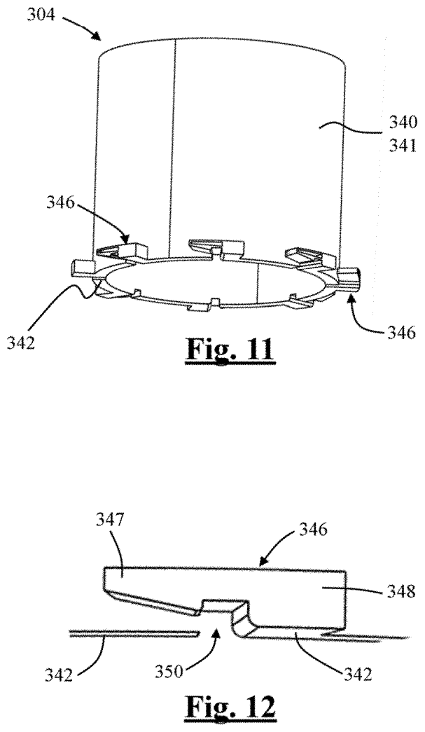

FIG. 11 illustrates the second or male part 304 of the coupling 300. The male part 304 comprises a hub 340. The hub 340 is defined proximal of a leading edge 342 of the male part. The leading edge 342 may be the distal-most end of the male part 304.

The male part 304 may comprise one or more retaining projections 346. The one or more retaining projections 346 may be disposed or extend from an outer surface 341 of the hub 340 and/or from the leading edge 342. The retaining projections 346 may be disposed radially around a circumference or perimeter of the hub 340. The retaining projections 346 may be equally spaced around the circumference or perimeter of the hub 340. Alternatively, the retaining projections 346 may be unequally spaced around the circumference or perimeter of the hub 340. Preferably, the retaining projections 346 are arranged or spaced on the hub 340 in a manner that generally or substantially corresponds to the arrangement or spacing of the receiving recesses 312 and/or retainers 314 on the female part 102 (FIGS. 10A-10B).

An inner portion of the hub 340 may include a rib or thread onto which the hose 12 or device 10 may be threaded or attached thereto. In some configurations, an outer surface of the hub 340 may include the rib or thread. In some configurations, the inner and outer surfaces of the hub 340 may include a rib or threads which would allow the male part 304 to connect with a variety of hose diameter or sizes.

Referring now to FIG. 12, each retaining projection 346 may include a leading end 347 and an opposing trailing end 348 that extends from the leading edge 342 of the hub 340. The retaining projection 346 includes a locking recess 350. The retaining projection 346 may be configured to flex or bend in a region of the trailing end 348 during assembly of the coupling 300.

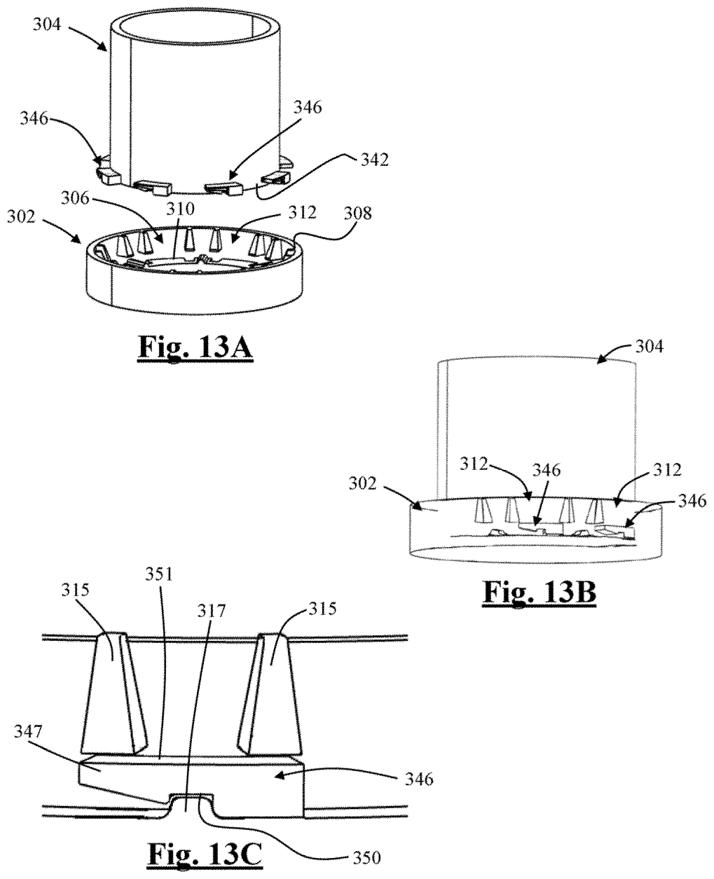

FIGS. 13A-13C illustrate assembling the coupler 300. It is understood that the following method steps can be performed in virtually any order. Moreover, one or more of the following method steps can be combined with other steps; can be omitted or eliminated; can be repeated; and/or can separated into individual or additional steps.

The leading edges 308, 342 of the female and male parts 302, 304 can be bought together so that the male part 304 is received into the female part 302 or into the socket 306 of the female part 302. More specifically, the female and male parts 302, 304 are bought together so that the retaining projections 346 on the male part 304 align with, and are then received into or within the corresponding receiving recesses 312 within the socket 306, as illustrated in FIG. 13B, which illustrates a portion of the female part 302 in transparent. This means that a length of the retaining projection 346 is at least slightly larger than the gap G′ between upper tabs 315 of adjacent retainers 314 (See also FIG. 10B). The parts 302, 304 may be brought together or towards each other until the leading edge 342 of the male part 304 is near, juxtaposed, bottoms out, abuts, or contacts the contact wall or ledge 310 of the female part 302.

One or both of the two parts 302, 304 can be rotated so that the leading end 347 of each of the one or more retaining projections 346 rides up and over a corresponding lower tab 317 of the retainer 314. While riding up the lower tab 317, the retaining projection 346 may bend or flex. Further rotation of one or both of the parts 302, 304 results in the lower tab 317 being positioned in the locking recess 350, defined between the trailing end 348 and the leading end 347. The two parts 302, 304 are thus rotationally locked together. Each of the upper tabs 315 are in contact with or immediately adjacent a top surface 351 of the retaining projection 346, thus restricting or preventing axial separation of the two parts 302, 304.

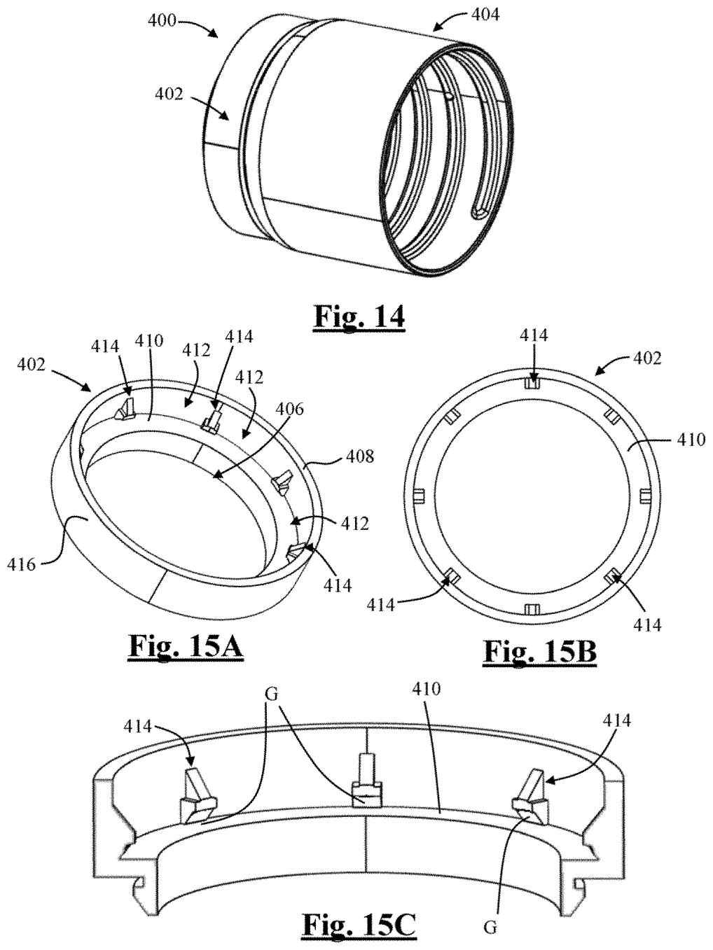

FIG. 14 illustrates a coupling 400 that may be used in place of the coupling 100. The coupling 400 includes a first or female part 402 and a second or male part 404. It is understood that one or more of the features illustrated and/or described relating to any of the couplings described herein (including those illustrated/described in couplings 100, 200, 300, etc.) may be incorporated into the coupling 400, and vice versa.

FIGS. 15A-15C illustrate the first or female part 402 of the coupling 400. The female part 402 comprises a socket 406. The socket 406 has a generally circular wall 416 that extends around a center of the female part 402. The socket 406 is defined between a leading edge 408 of the female part 402 and a contact wall or ledge 410. The leading edge 408 may be the distal-most end of the female part 402. The contact wall or ledge 410 extends inwardly from the wall 416 towards the center of the female part 402. A diameter defined by an inside of the contact wall or ledge 410 is smaller than a diameter defined by the generally circular wall 416.

The female part 402 may comprise one or more receiving recesses 412. The receiving recess 412 may be disposed or defined in the wall 416 of the socket 406. The receiving recesses 412 may be equally spaced around the circumference or perimeter of the socket 406. Alternatively, the receiving recesses 412 may be unequally spaced around the circumference or perimeter of the socket 406.

The female part 402 may comprise one or more retainers 414. The one or more retainers 414 may extend from the wall 416 or may be disposed, defined or formed in or the wall 416 of the socket 406. A retainer 414 may comprise one or more upper tabs, projections, or protuberances. There may be one or a plurality of the retainers 414 disposed around the socket 406, equally or unequally. Unequally as used throughout this disclosure may mean that the spacing between adjacent retainers 414 is not the same or is random. A gap G may be defined between the contact wall or ledge 410 and the retainer 414. A receiving recess 412 is a defined in a region or gap between two adjacent retainers 414.

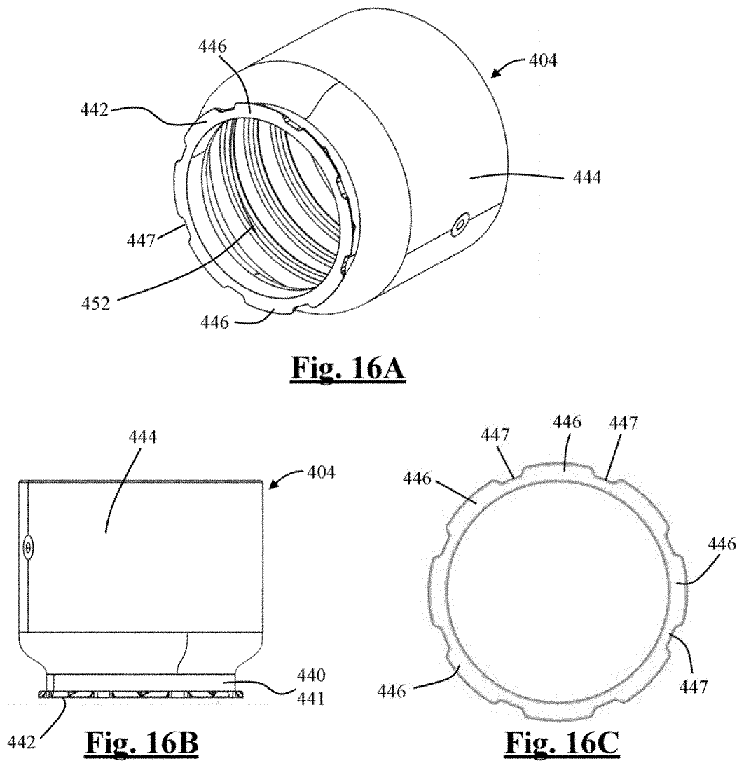

FIGS. 16A-16C illustrate the second or male part 404 of the coupling 400. The male part 404 comprises a hub 440. The hub 440 is defined proximal of a leading edge 442 of the male part 404. The leading edge 442 may be the distal-most end of the male part 404.

The male part 404 may comprise one or more retaining projections 446. The one or more retaining projections 446 may be disposed or radially extend from the leading edge 442. The retaining projections 446 may be disposed radially around a circumference or perimeter of the hub leading edge 442. The retaining projections 446 may be equally spaced around the circumference or perimeter of the hub 440. Alternatively, the retaining projections 446 may be unequally spaced around the circumference or perimeter of the hub 446. Preferably, the retaining projections 446 are arranged or spaced on the hub 440 in a manner that generally or substantially corresponds to the arrangement or spacing of the receiving recesses 412 and/or retainers 414 on the female part 402 (FIGS. 15A-15C). One or more recesses 447 may be defined between an adjacent pair of the retaining projections 446.

The male part 404 comprises a collar 444. The collar 444 may be the section of the male part 404 that connects with or engages one of the devices 10, 10′ or hose 12. An inner portion of the collar 444 may include a rib or thread 452 onto which the hose 12 or device may be threaded or attached thereto. In some configurations, an outer surface of the collar 444 may include the rib or thread. In some configurations, the inner and outer surfaces of the collar 444 may include a rib or threads which would allow the male part 404 to connect with a variety of hose diameter or sizes.

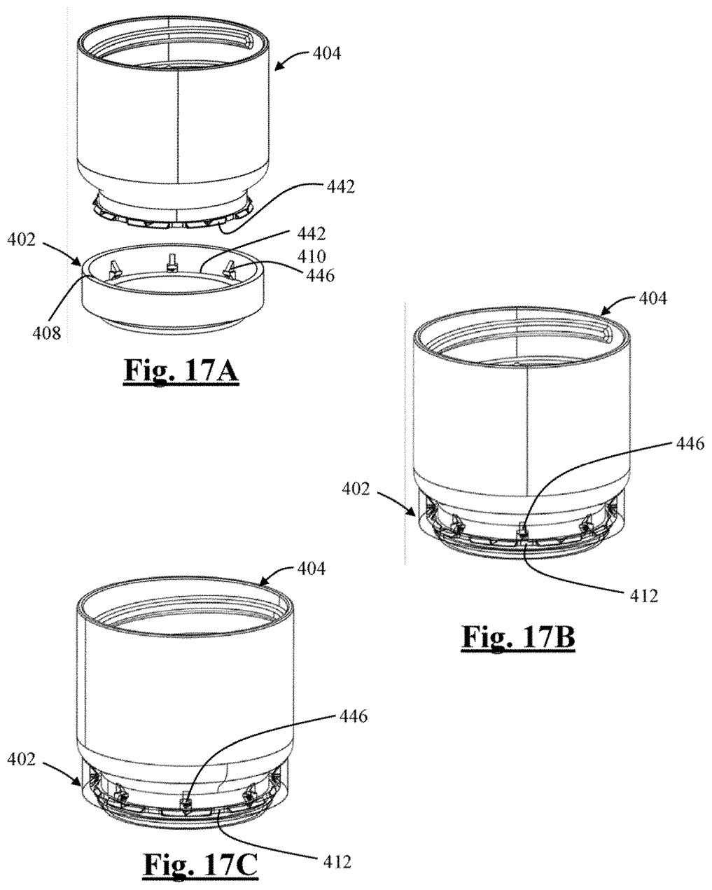

FIGS. 17A-17C illustrate assembling the coupler 400. It is understood that the following method steps can be performed in virtually any order. Moreover, one or more of the following method steps can be combined with other steps; can be omitted or eliminated; can be repeated; and/or can separated into individual or additional steps.

The leading edges 408, 442 of the female and male parts 402, 404 can be bought together so that the male part 404 is received into the female part 402 or into the socket 406 of the female part 402. More specifically, the female and male parts 402, 404 are bought together so that the retaining projections 446 on the male part 404 align with, and are then received into or within the corresponding receiving recesses 412 within the socket 406, as illustrated in FIG. 14B, which illustrates a portion of the female part 402 in transparent. The parts 402, 404 may be brought together or towards each other until the leading edge 442 of the male part 404 is near, bottoms out, abuts, or contacts the contact wall or ledge 410 of the female part 402.

One or both of the two parts 402, 404 can be rotated so that the one or more retaining projections 446 rides under a corresponding retainer 414. The two parts 402, 404 are thus rotationally and axially locked together.

The explanations and illustrations presented herein are intended to acquaint others skilled in the art with the invention, its principles, and its practical application. The above description is intended to be illustrative and not restrictive. Those skilled in the art may adapt and apply the invention in its numerous forms, as may be best suited to the requirements of a particular use.

Accordingly, the specific embodiments of the present invention as set forth are not intended as being exhaustive or limiting of the teachings. The scope of the teachings should, therefore, be determined not with reference to this description, but should instead be determined with reference to the appended claims, along with the full scope of equivalents to which such claims are entitled. The omission in the following claims of any aspect of subject matter that is disclosed herein is not a disclaimer of such subject matter, nor should it be regarded that the inventors did not consider such subject matter to be part of the disclosed inventive subject matter.

Plural elements or steps can be provided by a single integrated element or step. Alternatively, a single element or step might be divided into separate plural elements or steps.

The disclosure of “a” or “one” to describe an element or step is not intended to foreclose additional elements or steps.

While the terms first, second, third, etc., may be used herein to describe various elements, components, regions, layers and/or sections, these elements, components, regions, layers and/or sections should not be limited by these terms. These terms may be used to distinguish one element, component, region, layer or section from another region, layer or section. Terms such as “first,” “second,” and other numerical terms when used herein do not imply a sequence or order unless clearly indicated by the context. Thus, a first element, component, region, layer or section discussed below could be termed a second element, component, region, layer or section without departing from the teachings.

Spatially relative terms, such as “inner,” “outer,” “beneath,” “below,” “lower,” “above,” “upper,” and the like, may be used herein for ease of description to describe one element or feature's relationship to another element(s) or feature(s) as illustrated in the figures. Spatially relative terms may be intended to encompass different orientations of the device in use or operation in addition to the orientation depicted in the figures. For example, if the device in the figures is turned over, elements described as “below” or “beneath” other elements or features would then be oriented “above” the other elements or features. Thus, the example term “below” can encompass both an orientation of above and below. The device may be otherwise oriented (rotated 90 degrees or at other orientations) and the spatially relative descriptors used herein interpreted accordingly.

The disclosures of all articles and references, including patent applications and publications, are incorporated by reference for all purposes. Other combinations are also possible as will be gleaned from the following claims, which are also hereby incorporated by reference into this written description.

1) A coupling for connecting together a medical hose and a medical device, the coupling comprising:
a male part comprising a distal most end, and a retaining projection having a retaining surface located proximal to the distal-most end of the male part;
a female part comprising a retaining recess having a retaining top edge;
wherein the male part is configured to be received into the female part, and the retaining top edge of the retaining recess is configured to engage the retaining surface of the retaining projection, and
wherein the male part is connected to the medical hose and the female part is connected to the medical device, or the female part is connected to the medical hose and the male part is connected to the medical device,
wherein the medical device is a source of fluid, a thermal blanket, a pad, or a mattress.

2) The coupling according to claim 1, wherein the female part comprises a receiving recess,
wherein axial separation of the male part and the female part is permitted when the male part is received into the female part and the retaining projection is positioned within the receiving recess, and
wherein axial separation of the male part and the female part is restricted when the retaining projection is positioned within the retaining recess.

3) The coupling according to claim 1, wherein the retaining projection is moved between the receiving recess and the retaining recess by rotating the male part and/or the female part.

4) The coupling according to claim 1, wherein the retaining recess is defined between a clockwise edge and a counterclockwise edge, and
wherein the clockwise edge is arranged closer to a center of the female portion than the counterclockwise edge.

5) The coupling according to claim 1, wherein the receiving recess is arranged radially adjacent to the retaining recess.

6) The coupling according to claim 5, wherein the female part comprises a plurality of receiving recesses and a plurality of retaining recesses, and
wherein each retaining recesses is positioned between two adjacent receiving recesses.

7) The coupling according to claim 5, wherein the plurality of receiving recesses and the plurality of retaining recesses are spaced apart equally.

8) The coupling according to claim 1, wherein the female part comprises a socket having a socket contact wall, the distal-most end of the male part is configured to abut the socket contact wall.

9) The coupling according to claim 1, wherein the female part comprises a socket into which the male part is received, an outer surface of the socket is scalloped.

10) The coupling according to claim 1, wherein the female part comprises a distal-most end, and the retaining top edge is located proximal to the distal-most end of the female part.

11) The coupling according to claim 1, wherein the male part comprises a collar comprising one or more gripping projections.

12) The coupling according to claim 11, wherein the one or more gripping projections are generally aligned with the one or more retaining projections.

13) The coupling according to claim 1, wherein the male part comprises a proximal portion comprising a rib.

14) The coupling according to claim 13, wherein the rib is part of a thread.

15) The coupling according to claim 13, wherein the proximal portion comprises an undercut region.

16) The coupling according to claim 1, wherein the female part comprises a proximal portion comprising a rib.

17) An assembly comprising:
the coupling according to claim 1, the medical device, and the medical hose,
wherein the female part is connected to the medical device and the male part is connected to the medical hose.

18) The assembly according to claim 17, wherein the male part comprises a rib for threadably engaging the medical hose and an undercut region for at least a portion of the medical hose to engage.

19) The assembly according to claim 17, wherein the medical device is an air source.

20) The assembly according to claim 17, wherein the medical device is the thermal blanket.
